(19) United States
(12) Patent Application Publication
(10) Pub. No.: US 2025/0037277 A1
(43) Pub. Date: Jan. 30, 2025

(54) HIERARCHICAL MULTI-DISEASE DETECTION SYSTEM WITH FEATURE DISENTANGLEMENT AND CO-OCCURRENCE EXPLOITATION FOR RETINAL IMAGE ANALYSIS

(71) Applicant: AI Optics Inc., New York City, NY (US)

(72) Inventors: Luke Michael Moretti, New York City, NY (US); Benjamin JJ Villard, Charleston, SC (US)

(21) Appl. No.: 18/755,153

(22) Filed: Jun. 26, 2024

Related U.S. Application Data
(60) Provisional application No. 63/515,457, filed on Jul. 25, 2023.

(51) Int. Cl. G06T 7/00; G16H 30/40

(52) U.S. Cl. CPC G06T 7/0012; G16H 30/40; G06T 2207/20076; G06T 2207/20081; G06T 2207/20084; G06T 2207/30041

(57) ABSTRACT

Systems and methods for accurate, reliable, and comprehensive multi-disease retinal screening is disclosed. Leveraging a hierarchical deep learning architecture, a system can optimize feature extraction through a convolutional neural network (CNN) by first classifying images as normal or abnormal, and then discern the presence and severity of specific diseases using disease-specific CNN branches. The hierarchical deep learning architecture can promote feature disentanglement by associating each branch with a potentially unique set of parameters, enhancing disease-specific feature sensitivity. The system can further refine disease detection accuracy by implementing a probability classifier to exploit patterns of disease co-occurrence. A Monte Carlo Expectation-Maximization algorithm can optimize the parameters of the probability classifier, and a subsequent prediction calibration step can correct potential biases in the final predictions. An end-to-end learning approach using backpropagation to optimize all parameters concurrently can be employed to ensure robust disease prediction outcomes.

### CROSS-REFERENCE TO RELATED APPLICATIONS

This application claims priority to U.S. Provisional application 63/515,457, filed Jul. 25, 2023, titled “Hierarchical Multi-Disease Detection System With Feature Disentanglement And Co-Occurrence Exploitation For Retinal Image Analysis,” which is incorporated by reference in its entirety.

### TECHNICAL FIELD

This disclosure relates to the field of medical image analysis, particularly using deep learning models to detect multiple diseases in retinal images.

### BACKGROUND

Retinal diseases, including diabetic retinopathy, age-related macular degeneration, and glaucoma, among others, are leading causes of vision loss globally. Early detection and treatment can prevent or significantly delay vision loss. Digital retinal imaging using fundus cameras is a non-invasive method that enables detailed examination of the retina and, when combined with telemedicine, allows for remote diagnosis and monitoring of retinal diseases. Still, there exist several challenges with the current systems and methods of medical imaging, which can be resolved using the approaches described herein.

### SUMMARY

The systems, methods, and devices described herein each have several aspects, no single one of which is solely responsible for its desirable attributes. Without limiting the scope of this disclosure, several non-limiting features will now be discussed briefly.

A method for disease detection using retinal images can include processing a retinal image of a retina with a first machine learning model to determine whether the retinal image reflects an abnormal condition or a normal condition of the retina. The method can include, at a first time, determining that the retinal image reflects the abnormal condition of the retina and in response to determining that the retinal image reflects the abnormal condition of the retina, processing the retinal image by a plurality of second machine learning models. Each machine learning model of the plurality of second machine learning models can be configured to identify 1) a presence of a disease different from any other machine learning model of the plurality of second machine learning models and 2) a severity of the disease. The method can include processing a plurality of disease presences and a plurality of disease severities determined by the plurality of second machine learning models with a third machine learning model to determine a plurality of disease indicators based on relationships between a plurality of diseases and their respective severities identified by the plurality of second machine learning models. The method can include outputting the plurality of disease indicators. The method can include outputting the retinal image. The method can include, at a second time, determining that the retinal image reflects the normal condition of the retina and in response to determining that the retinal image reflects the normal condition of the retina, outputting the retinal image. The method can be performed by one or more processors.

The method of any of the preceding paragraphs and/or any of the methods described herein can include one or more of the following features.

The relationships between the plurality of diseases can include a disease co-occurrence pattern. The third machine learning model can include a graph-based machine learning model configured to determine the plurality of disease indicators using the disease co-occurrence pattern.

The graph-based machine learning model can include conditional random fields.

The plurality of disease indicators can be determined by generating a plurality of nodes, wherein each node of the plurality of nodes is based on a plurality of detection probabilities for the plurality of disease presences and a plurality of severity estimation probabilities for the plurality of disease severities, determining one or more neighboring nodes for each node of the plurality of nodes based on the disease co-occurrence pattern, and updating a detection probability and a severity estimation probability for each disease of the plurality of diseases based on the plurality of detection probabilities and plurality of severity estimation probabilities for neighboring nodes, the plurality of disease indicators including the updated detection probabilities and the updated severity estimation probabilities.

The first machine learning model can include a binary classification convolutional neural network configured to differentiate between abnormal and normal conditions of the retina.

The plurality of second machine learning models can include a plurality of multi-branch disease-specific convolutional neural networks.

Each machine learning model of the plurality of second machine learning models can include a disease detection branch and a disease severity branch that is separate from the disease detection branch.

The disease detection branch can be configured to determine a detection probability for the presence of the disease, and the disease severity branch can be configured to determine a severity estimation probability for the severity of the disease.

The disease detection branch and the disease severity branch can be configured to minimize a loss function using backpropagation to reduce a combination of the loss function for the disease detection branch and the loss function of the disease severity branch.

The third machine learning model can be configured to minimize a loss function that measures a discrepancy between a predicted co-occurrence pattern and an actual co-occurrence pattern, and the loss function for the third machine learning model can include a co-occurrence loss function.

The loss function of the disease detection branch can include a binary cross-entropy loss function and the loss function of the disease severity branch includes a categorical cross-entropy loss function.

The method can include minimizing a total loss determined based on a weighted sum of weights for each of the loss functions and updated weights determined based on a validation performance for each of the loss functions.

Updated weights can be determined based on backpropagating an error signal corresponding to each of the loss functions to minimize the error signal.

The method can include optimizing the plurality of second machine learning models using backpropagation and optimizing the third machine learning model using an expectation maximization.

Outputting the plurality of disease indicators can include outputting probabilities of the plurality of disease severities.

Outputting probabilities of the plurality of disease severities can include outputting an ordered representation of probabilities of the plurality of disease severities.

Outputting probabilities of the plurality of disease severities can include outputting a level of severity for a plurality of diseases based on a likelihood or frequency of disease occurrence.

### BRIEF DESCRIPTION OF DRAWINGS

Various aspects of the disclosure will now be described with regard to certain examples and implementations, which are intended to illustrate but not limit the disclosure.

Throughout the drawings, reference numbers may be re-used to indicate correspondence between referenced elements. The drawings are provided to illustrate example implementations described herein and are not intended to limit the scope of the disclosure.

FIG. 1 illustrates an example retina camera.

FIG. 2 illustrates a block diagram of various components of the retina camera.

FIG. 3 illustrates a block diagram of a hierarchical deep learning architecture.

FIG. 4 illustrates a block diagram of a single disease classifier.

FIG. 5 illustrates a block diagram of a disease classifier processing the initial disease probabilities to determine refined diseases probabilities.

FIGS. 6A and 6B illustrate example user interfaces of a results display.

### DETAILED DESCRIPTION

Implementations of disclosed systems and methods relate to a hierarchical multi-disease detection system with biomarker identification and co-occurrence exploitation for retinal image analysis, which can address the limitations of existing methods in the field of automated retinal disease detection. The system is designed to improve upon traditional deep learning methods by incorporating a hierarchical learning structure, sophisticated biomarker identification, and disease co-occurrence exploitation.

### Overview

In many regions, especially those with limited healthcare resources, screening for retinal diseases can be inadequate, and as a result, patients can be diagnosed at the late stages of the disease when irreversible vision loss has already occurred. Early detection and treatment can prevent or significantly delay vision loss.

Digital retinal imaging using fundus cameras is a non-invasive method that enables detailed examination of the retina and, when combined with telemedicine, allows for remote diagnosis and monitoring of retinal diseases. Despite the advantages of retinal imaging using a fundus camera, human interpretation of retinal images can be time-consuming, can require skilled ophthalmologists or optometrists, and can be subject to inter-observer and intra-observer variability.

Hence, there is a significant interest in developing automated retinal image analysis tools that can provide consistent and reliable screening for multiple retinal diseases. As a result, there has been significant interest in automated retinal image analysis, which can utilize computer vision and machine learning technologies. Particularly, deep learning-based methods, which can automatically learn to extract and use features from images can be useful for retinal image analysis. Deep learning-based methods for retinal disease detection can fall into one of two categories: single-task or multi-task models.

Single-task models can be designed to detect a specific disease from retinal images. For example, an algorithm might be trained exclusively for diabetic retinopathy detection using large, labeled datasets of retinal images. This can be achieved using a Convolutional Neural Network (CNN), a deep learning architecture well-suited to image data, which can learn to identify disease-specific features from the input images. The CNN can be trained end-to-end to map input images to disease labels, learning from thousands or even millions of labeled examples.

Multi-task models can aim to detect multiple diseases from retinal images simultaneously. For example, the output branch or layer of the CNN can be modified to produce multiple outputs, where each output can correspond to a different disease. For example, a model might be trained to output probabilities of both diabetic retinopathy and glaucoma. Multi-task models can leverage shared features between different diseases, potentially improving efficiency and performance.

Developing deep learning models for multi-disease detection from medical images can involve several meaningful technical challenges limiting their utility and value. A few potential challenges and limitations are described below.

Insufficient Inter-Disease and Intra-Disease Feature Disentanglement: Handling variations within and between diseases can pose a significant challenge due to the complexity and diversity of disease presentations in medical imaging. When a model learns to distinguish multiple diseases (inter-disease), the model can learn unique biomarkers that can separate one disease from another. However, in medical images, multiple diseases can present similar visual cues or can have features indicative of disease overlap with one another. For example, in retinal imaging both diabetic retinopathy and retinal vein occlusion can lead to hemorrhages. Designing and training a model that can effectively extract and understand these subtle differences can present a challenge as it can require well-curated data with a variety of examples for each disease and sophisticated network architectures that can learn complex feature representations. Additionally, different patients can exhibit different retinal image biomarkers even when suffering from the same disease (intra-disease variance). Similarly, the same disease can evolve differently within the same patient over time. For example, early-stage glaucoma can be subtle with minimal cupping of the optic nerve head, while late-stage glaucoma can have a more pronounced cupping. Variation within the same disease can make the feature learning process for the model challenging, as the model may need to recognize a wide range of disease presentations while being flexible enough to understand the progression of a single disease. Extracting and disentangling these features can be a major challenge and existing methods can fail to account for these variations sufficiently, which can lead to suboptimal performance.

One approach may be to train a model on a vast dataset hoping that the diversity in the data will lead the model to learn these variations. However, this approach has several potential drawbacks. First, even with large datasets, certain patterns or correlations may not be well represented or could be missed altogether, which can lead to poor generalization on unseen data that contains these less frequent or missing patterns. Instead, a hierarchical deep learning architecture can provide for learning at different levels of granularity, which can help the model understand the smaller, more subtle patterns in the data in addition to broader ones. Additionally, by integrating a probability classifier, such as a Gaussian Conditional Random Field (G-CRF) to model disease co-occurrence, this approach can effectively capture and utilizes the correlations between diseases that might not be well represented or could be missed in a vast dataset. Furthermore, and importantly, gathering data on rare or less common retinal diseases presents significant challenges because rare diseases often have fewer diagnosed cases, making it harder to collect a large and diverse dataset. The rarity of these diseases can also make it difficult to obtain a dataset that represents a wide variety of patient characteristics like age, sex, race, and geography, which can be important for building a model that is equitable and universally applicable. Additionally, the disease presentations for rare diseases can often vary widely between patients, which means a larger, more diverse dataset may be needed to capture the full spectrum of disease manifestations and related retinal features. Given the constraints and precautions around HIPPA-compliant data sharing, obtaining the data needed is a major practical challenge.

The present disclosure bypasses these limitations in several significant ways. First, the hierarchical deep learning architecture can be particularly effective by learning to recognize broad patterns of disease in the first stage, patterns which may be common across a range of diseases, both common and rare. Then, in the second stage, the model can learn to distinguish between specific diseases. This two-stage learning process means the model can learn to recognize a rare disease based on its similarities and differences with more common diseases, and effectively leveraging the information from all available data. Additionally, the approach used in this system involves multi-task learning, where the model can learn to perform two tasks simultaneously, disease detection and disease severity estimation. Multi-task learning can allow for even a small amount of data on a rare disease to be used more effectively, as the model can learn from the data for both tasks at once. Furthermore, the use of a third stage, such as G-CRF, for modeling disease co-occurrence patterns can help to compensate for limited data by allowing the model to understand and leverage the relationships between different diseases. For example, if a rare disease often co-occurs with a more common disease, the model can use its understanding of the common disease to help predict the presence of the rare disease. These components allow the model to make the most out of the available data, effectively learning to recognize and assess both common and rare retinal diseases.

Disease Co-Occurrence: A challenge for developing a multi-disease retinal imaging diagnostic system can be due to a retinal image exhibiting signs of more than one disease, and therefore belonging to more than one class. This situation can pose a multi-label classification problem, which can be a more complex task than single-label classification. An improvement using hierarchical model approaches described herein can be that that the model may not only identify which diseases are present but may also make sense of correlations or dependencies between different labels. For example, a patient with diabetes can be more likely to have diabetic retinopathy and glaucoma, but less likely to have a condition unrelated to diabetes. Many existing methods may not specifically consider these disease co-occurrence patterns, which can lead to less accurate predictions, particularly in cases where multiple diseases are present.

Lack of Hierarchical Learning: Existing methods can often treat disease detection as a flat, multi-label classification problem. This approach fails to consider the inherent hierarchy of normal and abnormal conditions, such as an image being abnormal as a prerequisite for it having a specific disease. In comparison, incorporating hierarchical learning approached disclosed herein can allow a model to first determine whether an image is normal or abnormal, and then decide which specific disease it represents. This can improve computational efficiency and can enable the model to learn more discriminative features.

Disclosed hierarchical model approaches for multi disease classification can solve at least the above challenges and limitations of existing techniques and, thereby, improve the accuracy and reliability of automated retinal disease screening.

### Machine Learning Models

To facilitate an understanding of the systems and methods discussed herein, several terms are described below. These terms, as well as other terms used herein, should be construed to include the provided descriptions, the ordinary and customary meanings of the terms, and/or any other implied meaning for the respective terms, wherein such construction is consistent with context of the term. Thus, the descriptions below do not limit the meaning of these terms, but only provide example descriptions.

The term “model,” as used in the present disclosure, can include any computer-based model of any type and of any level of complexity, such as any type of sequential, functional, or concurrent model. Furthermore, although certain implementations of the disclosure refer to CNNs, this is not intended to be limiting and the disclosed approaches can include other models in conjunction with or in lieu of. Models can further include various types of computational models, such as, for example, artificial neural networks (“NN”), language models (such as, large language models (“LLMs”)), artificial intelligence (“Al”) models, machine learning (“ML”) models, multimodal models (e.g., models or combinations of models that can accept inputs of multiple modalities, such as images and text), and/or the like.

While certain aspects and implementations are discussed herein with reference to use of a language model, CNN, and/or artificial intelligence (AI), those aspects and implementations may be performed by any other language model, CNN, Al model, generative Al model, generative model, ML model, NN, multimodel model, and/or other algorithmic processes. Similarly, while certain aspects and implementations are discussed herein with reference to use of a ML model, those aspects and implementations may be performed by any other Al model, generative Al model, generative model, NN, multimodel model, and/or other algorithmic processes.

CNN and/or other models (including ML models) of disclosed implementations may be locally hosted, cloud managed, accessed via one or more Application Programming Interfaces (“APIs”), and/or any combination of the foregoing and/or the like. Additionally, in various implementations, the CNNs and/or other models (including ML models) of may be implemented in or by electronic hardware such application-specific processors (e.g., application-specific integrated circuits (“ASICs”)), programmable processors (e.g., field programmable gate arrays (“FPGAs”)), application-specific circuitry, and/or the like. Data that may be queried using the systems and methods of the present disclosure may include any type of electronic data, such as text, files, documents, books, manuals, emails, images, audio, video, databases, metadata, positional data (e.g., geo-coordinates), geospatial data, sensor data, web pages, time series data, and/or any combination of the foregoing and/or the like. In various implementations, such data may comprise model inputs and/or outputs, model training data, modeled data, and/or the like.

Medical Diagnostics Devices with On-Board AI

A device with integrated artificial intelligence (AI) can be used to assess a patient's body part to detect a disease. The device can be portable or handheld by a user (which can be a patient or a healthcare provider). For example, the device can be a retina camera configured to assess a patient's eye (or retina) and, by using an on-board Al retinal disease detection system, provide real-time analysis and diagnosis of disease that caused changes to the patient's retina. Easy and comfortable visualization of the patient's retina can be facilitated using such retina camera, which can be placed over the patient's eye, display the retina image on a high-resolution display, potentially with screenshot capabilities, analyze a captured image by the on-board Al system, and provide determination of presence of a disease.

Such retina camera can perform data collection, processing, and diagnostics tasks on-board without the need to connect to another computing device or to cloud computing services. This approach can avoid potential interruptions of the clinical workflow when using cloud-based solutions, which involve transfer of data over the network and, accordingly, rely on network connectivity. This approach can facilitate faster processing because the device can continually acquire and process images without needing intermediary upload/download steps, which can be slow. Such retina camera can potentially improve accuracy (for instance, as compared to retina cameras that rely on a human to perform analysis), facilitate usability (for example, because no connectivity is used to transfer data for analysis or transfer results of the analysis), provide diagnostic results in real-time, facilitate security and guard patient privacy (for example, because data is not transferred to another computing device), or the like. Such retina camera can be used in many settings, including places where network connectivity is unreliable or lacking.

Such retina camera can allow for better data capture and analysis, facilitate improvement of diagnostic sensitivity and specificity, and improve disease diagnosis in patients. Existing fundus cameras can lack one or more of portability, display, on-board Al capabilities, etc. or require one or more of network connectivity for sharing data, another device (such as, mobile phone or computing device) to view collected data, rigorous training of the user, etc. In contrast, allowing for high-quality retinal viewing and image capturing with faster analysis and detection of the presence of disease via on-board Al system and image-sharing capabilities, the retina cameras described herein can potentially provide improved functionality, utility, and security. Such retina camera can be used in hospitals, clinics, and/or at home. The retina cameras or other instruments described herein, however, need not include each of the features and advantages recited herein but can possibly include any individual one of these features and advantages or can alternatively include any combination thereof.

As another example, the device can be an otoscope configured to assess a patient's ear and, by using an on-board artificial intelligence (AI) ear disease detection system, possibly provide immediate analysis and/or diagnosis of diseases of the patient's ear. Such an otoscope can have one or more advantages described above or elsewhere in this disclosure. As yet another example, the device can be a dermatology scope configured to assess a patient's skin and, by using an on-board artificial intelligence (AI) skin disease detection system, possibly provide immediate analysis and/or diagnosis of diseases of the patient's skin. Such a dermatology scope can have one or more advantages described above or elsewhere in this disclosure.

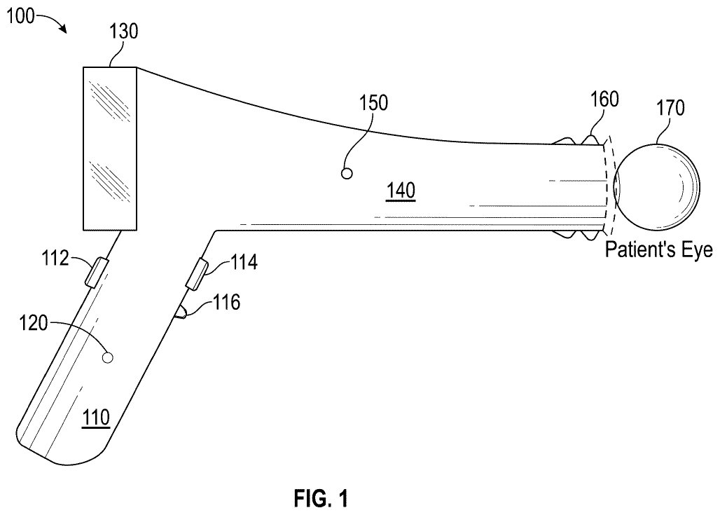

FIG. 1 illustrates an example retina camera 100. A housing of the retina camera 100 can include a handle 110 and a body 140 (in some cases, the body can be barrel-shaped). The handle 110 can optionally support one or more of power source, imaging optics, or electronics 120. The handle 110 can also possibly support one or more user inputs, such as a toggle control 112, a camera control 114, an optics control 116, or the like. Toggle control 112 can be used to facilitate operating a display 130 in case of a malfunction. For example, toggle control 112 can facilitate manual scrolling of the display, switching between portrait or landscape mode, or the like. Toggle control 112 can be a button. Toggle control 112 can be positioned to be accessible by a user's thumb. Camera control 114 can facilitate capturing video or an image. Camera control 114 can be a button. Camera control 114 can be positioned to be accessible by a user's index finger (such as, to simulate action of pulling a trigger) or middle finger. Optics control 116 can facilitate adjusting one or more properties of imaging optics, such as illumination adjustment, aperture adjustment, focus adjustment, zoom, etc. Optics control 116 can be a button or a scroll wheel. For example, optics control 116 can focus the imaging optics. Optics control 116 can be positioned to be accessible by a user's middle finger or index finger.

The retina camera 100 can include the display 130, which can be a liquid crystal display (LCD) or other type of display. The display 130 can be supported by the housing as illustrated in FIG. 1. For example, the display 130 can be positioned at a proximal end of the body 140. The display 130 can be one or more of a color display, high resolution display, or touch screen display. The display 130 can reproduce one or more images of the patient's eye 170. The display 130 can allow the user to control one or more image parameters, such as zoom, focus, or the like. The display 130 (which can be a touch screen display) can allow the user to mark whether a captured image is of sufficient quality, select a region of interest, zoom in on the image, or the like. Any of the display or buttons (such as, controls, scroll wheels, or the like) can be individually or collectively referred to as user interface. The body 140 can support one or more of the power source, imaging optics, imaging sensor, electronics 150 or any combination thereof.

A cup 160 can be positioned on (such as, removably attached to) a distal end of the body 140. The cup 160 can be made at least partially from soft and/or elastic material for contacting patient's eye orbit to facilitate examination of patient's eye 170. For example, the cup can be made of plastic, rubber, rubber-like, or foam material. Accordingly, the cup 160 can be compressible. The cup 160 can also be disposable or reusable. In some cases, the cup 160 can be sterile. The cup 160 can facilitate one or more of patient comfort, proper device placement, blocking ambient light, or the like. Some designs of the cup can also assist in establishing proper viewing distance for examination of the eye and/or pivoting for panning around the retina.

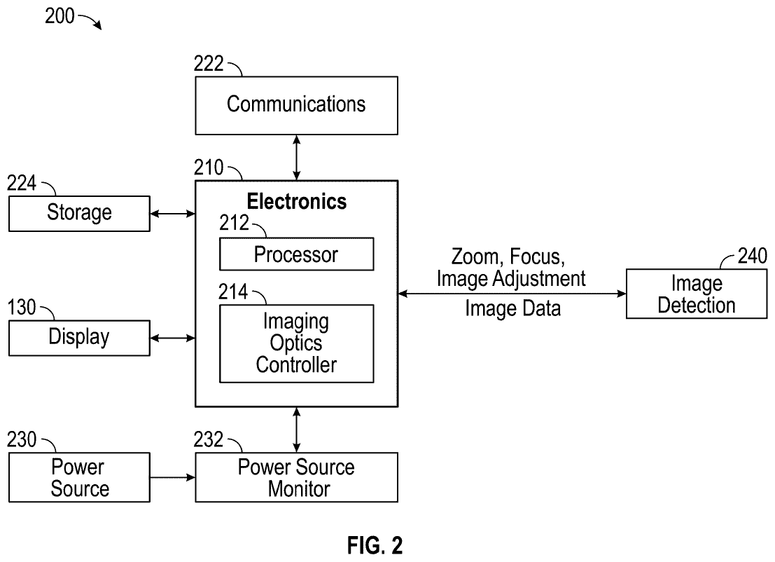

FIG. 2 illustrates a block diagram 200 of various components of the retina camera 100. Power source 230 can be configured to supply power to electronic components of the retina camera 100. Power source 230 can be supported by the handle 110, such as positioned within or attached to the handle 110 or be placed in another position on the retina camera 100. Power source 230 can include one or more batteries (which can be rechargeable). Power source 230 can receive power from a power supply (such as, a USB power supply, AC to DC power converter, or the like). Power source monitor 232 can monitor level of power (such as, one or more of voltage or current) supplied by the power source 230. Power source monitor 232 can be configured to provide one or more indications relating to the state of the power source 230, such as full capacity, low capacity, critical capacity, or the like. One or more indications (or any indications disclosed herein) can be visual, audible, tactile, or the like. Power source monitor 232 can provide one or more indications to electronics 210.

Electronics 210 can be configured to control operation of the retina camera 100. Electronics 210 can include one or more hardware circuit components (such as, one or more controllers or processors 212), which can be positioned on one or more substrates (such as, on a printed circuit board). Electronics 210 can include one or more of at least one graphics processing unit (GPU) or at least one central processing unit (CPU). Electronics 210 can be configured to operate the display 130. Storage 224 can include memory for storing data, such as image data obtained from the patient's eye 170, one or more parameters of Al detection, or the like. Any suitable type of memory can be used, including volatile or non-volatile memory, such as RAM, ROM, magnetic memory, solid-state memory, magnetoresistive random-access memory (MRAM), or the like. Electronics 210 can be configured to store and retrieve data from the storage 224.

Communications system 222 can be configured to facilitate exchange of data with another computing device (which can be local or remote). Communications system 222 can include one or more of antenna, receiver, or transmitter. In some cases, communications system 222 can support one or more wireless communications protocols, such as WiFi, Bluetooth, NFC, cellular, or the like. In some instances, the communications system can support one or more wired communications protocols, such as USB. Electronics 210 can be configured to operate communications system 222. Electronics 210 can support one or more communications protocols (such as, USB) for exchanging data with another computing device.

Electronics 210 can control one or more imaging devices 240, which can be configured to facilitate capturing of (or capture) image data of the patient's eye 170. Electronics 210 can control one or more parameters of the imaging devices 240 (for example, zoom, focus, aperture selection, image capture, provide image processing, or the like). Such control can adjust one or more properties of the image of the patient's eye 170. Electronics 210 can include an imaging optics controller 214 configured to control one or parameters of the imaging devices 240. Imaging optics controller 214 can control, for example, one or more motor drivers of the imaging devices 240 to drive motors (for example, to select an aperture, to select lenses that providing zoom, to move of one or more lenses to provide autofocus, to move a detector array or image sensor to provide manual focus or autofocus, or the like). Control of one or more parameters of the imaging devices 240 can be provided by one or more of user inputs (such as a toggle control 112, a camera control 114, an optics control 116, or the like), display 130, etc. Imaging devices 240 can provide image data (which can include one or more images) to electronics 210. As disclosed herein, electronics 210 can be supported by the retina camera 100. Electronics 210 can not be configured to be attached to (such as, connected to) another computing device (such as, mobile phone or server) to perform determination of presence of a disease.

### Disease Identification Through Image Analysis

Electronics 210 can include one or more controllers or processors (such as, a processor 212), which can be configured to analyze one or more images to identify a disease. For example, electronics 210 can include a processing system (such as, a Jetson Nano processing system manufactured by NVIDIA or a Coral processing system manufactured by Google), a System-on-Chip (SoC), or a Field-Programmable Gate Array (FPGA) to analyze one or more images. One or more images (or photographs) or video can be captured, for example, by the user operating the camera control 114 and stored in the storage 224. One or more prompts can be output on the display 130 to guide the user (such as, “Would you like to capture video or an image?”). Additionally or alternatively, symbols and graphics can be output on the display 130 to guide the user. Image quality can be verified before or after processing the one or more images or storing the one or more images in the storage 224. If any of the one or more images is determined to be of poor quality (for instance, as compared to a quality threshold), the image can not be processed or stored, the user can be notified, or the like. Image quality can be determined based on one or more of brightness, sharpness, contrast, color accuracy, distortion, noise, dynamic range, tone reproduction, or the like.

One or more preset modes can facilitate easy and efficient capture of multiple images or video. Such one or more preset modes can automatically focus, capture, verify image quality, and store the video or image(s). For some designs the one or more preset modes can switch one or more settings (such as, switch the light source to infrared light), and repeat this cycle without user intervention. In some designs, for example, a preset mode can facilitate obtaining multiple images for subsequent analysis. Such multiple images, for example, can be taken from different angles, use different light sources, or the like. This feature can facilitate automatically collecting an image set for the patient.

The user can select a region of an image for analysis, for instance, by outlining the region on the touch screen display 130, zooming in on region of interest on the display 130, or the like. In some cases, by default the entire image can be analyzed.

One or more machine learning models (sometimes referred to as Al models) can be used to analyze one or more images or video. One or more machine learning models can be trained using training data that includes images or video of subjects having various diseases of interest, such as retina disease (retinopathy, macular degeneration, macular hole, retinal tear, retinal detachment, or the like), ocular disease (cataracts or the like), systemic disease (diabetes, hypertension, or the like), Alzheimer's disease, etc. For example, any of the machine learning models can include a convolution neural network (CNN), decision tree, support vector machine (SVM), regressions, random forest, or the like. One or more machine learning models processing such images or videos can be used for tasks such as classification, prediction, regression, clustering, reinforcement learning, dimensionality reduction. Training of one or more models can be performed using many annotated images or video (such as, thousands of images or videos, tens of thousands of images or videos, hundreds of thousands of images or videos, or the like). Training of one or more models can be performed external to the retina camera 100. Parameters of trained one or more machine learning models (such as, model weights) can be transferred to the retina camera, for example, via retina camera's wireless or wired interface (such as, USB interface). Parameters of one or more models can be stored in the storage 224 (or in another memory of electronics 210). Output of the analysis (sometimes referred to as a diagnostic report) can include one or more of determination of the presence of disease(s), severity of disease(s), character of disease(s), clinical recommendation(s) based on the likelihood of presence or absence of disease(s). A diagnostic report can be displayed on the display 130. The diagnostic report can be stored in electronic medical record (EMR) format, such as EPIC EMR, or other document format (for example, PDF). The diagnostic report can be transmitted to a computing device. In some cases, the diagnostic report but not image data can be transmitted to the computing device, which can facilitate compliance with applicable medical records regulations (such as, HIPPA, GDPR, or the like).

One or more machine learning models can determine the presence of a disease based on the output of one or more models satisfying a threshold. As described herein, images or videos can be analyzed by one or more machine learning models one at a time or in groups to determine presence of the disease. For instance, the threshold can be 90%. When images are analyzed one at a time, determination of presence of the disease can be made in response to output of one or more models satisfying 90%. When images are analyzed in a group, determination of presence of the disease can be made in response to combined outputs of one or more models analyzing the group of images satisfying 90%.

In addition to these machine learning models, an Explainable Al framework (XAI) can be used to enhance the transparency and interpretability of the disease identification process. The XAI model can provide clear, understandable reasoning behind the Al model's decisions, enhancing trust in the system and facilitating the medical practitioner's understanding of the basis for the AI's disease identification. The explanatory components can include visual saliency maps indicating areas of importance, textual explanations highlighting key features leading to the prediction, or a combination of both. The explain ability feature can also help in identifying bias in Al model predictions, ensuring the robustness of the system.

The user can provide information (or one or more tags) to increase accuracy of the analysis by one or more machine learning models. For example, the user can identify any relevant conditions, symptoms, or the like that the patient (and/or one or more patient's family members) has been diagnosed with or has experienced. Relevant conditions can include systemic disease, retinal disease, ocular disease, or the like. Relevant symptoms can include blurry vision, vision loss, headache, or the like. Symptom timing, severity, or the like can be included in the identification. The user can provide such information using one or more user interface components on the display 130, such as a drop-down list or menu. One or more tags can be stored along with one or more pertinent images in the storage 224. One or more tags can be used during analysis by one or more machine learning models during analysis and evaluation. One or more images along with one or more tags can be used as training data.

In some cases, the diagnostic report can alternatively or additionally provide information indicating increased risk of disease or condition for a physician's (such as, ophthalmologist's) consideration or indicating the presence (or absence) of disease of condition. Physician can use this information during subsequent evaluation of the patient. For example, the physician can perform further testing to determine if one or more diseases are present.

Image or video analysis, including the application of one or more machine learning models to one or more images or video, can be performed by execution of program instructions by a processor and/or by a specialized integrated circuit that implements the machine learning model in hardware.

Disclosed devices and methods can, among other things, make the process of retinal assessment comfortable, easy, efficient, and accurate. Disclosed devices and methods can be used in physician offices, clinics, emergency departments, hospitals, in telemedicine setting, or elsewhere. Unnecessary visits to a specialist healthcare provider (such as, ophthalmologist) can be avoided, and more accurate decisions to visit a specialist healthcare provider can be facilitated. In places where technological infrastructure (such as, network connectivity) is lacking, disclosed devices and methods can be used because connectivity is not needed to perform the assessment.

### Video Capture and Analysis

In an example, every frame in a retinal video feed can be analyzed. In real-time, each frame can be fed through the image quality assessment and, subsequently, through a feature, disease, or condition detection (which can be implemented as one or more Al models). As another example, instead of individual frame analysis, the system could implement the concept of frame pooling where a defined group of frame is analyzed together. This group analysis could potentially identify subtle changes or patterns that might be missed when analyzing frames in isolation. The frames can be selected by taking into consideration the temporal, or sequential, position of the frames. Using the time-series information in addition to the information contained within the image data (such as, pixels) of the frame can increase the robustness of the one or more Al models. For example, for a given video of 5,000 frames, analysis can be performed in such a way that it: a) considers all 5,000 frames sequentially, b) considers a subset of the frames (such as, every other frame, groups of 10 frames or less of more, every 30th frame such that a frame is considered every minute for a video that includes 30 frames per second, or the like), while keeping the order, c) considers a subset of the frames with order being irrelevant (taking advantage of the knowledge that the frames belong to a times-series), or d) considers all frames as individual images, foregoing any temporal information and basing its resulting output on whether one or more features, diseases, or conditions are present in any particular frame. Those frames whose quality has been determined to be sufficient (such as, satisfying one or more thresholds) can be provided to the feature, disease, or condition detection.

In some implementations, one or more frames can undergo the feature, disease, or condition detection provided that the one or more frames have successfully passed the first step of image quality assessment (for instance, the verification that they are of sufficient quality). In some cases, disease, condition, or feature detection can be performed once the video (or live feed) is in focus, within a specific brightness range, absent of artifacts (such as, reflections or blurring), or the like. This verification can be performed before or after any preprocessing (such as, brightness adjustments or the like). For example, once there is a clear, in-focus view of the retina, the Al can automatically start analyzing frames for detection of features, diseases, or conditions. In some cases, if the video or live feed goes out of focus, the analysis for features, diseases, or conditions can cease until the video is back in focus. The image quality assessment that analyzes whether the device is in-focus (or absent of artifacts, etc.) can be separate (such as, separate processing or a module) from the detection of features, disease, or conditions. The image quality assessment that analyzes whether the device is in focus can display or relay information to the user to help improve the focus.

There can be processing or a module (which can be separate from or part of the image quality assessment) that aids in the maintenance of focus or specific video or frame characteristics (such as, brightness, artifacts, etc.). For example, once the retina comes into focus, there can be a software or hardware module that automatically adjusts the focus of the image and/or imaging optics to maintain the focused retinal image. Assessment of the movement during the video recording process can be performed and correction for the motion can be made, for example, by using a machine learning (ML) model that processes the captured images.

An indication can be provided to the user when the video (or frames) is of sufficient quality based on the image quality assessment. The indication can be one or more of visual, audible, tactile, or the like. For example, a green ring (or another indication) can appear around the outside edge of the retinal video feed when the frames (such as, any of the frames from a group of frames or all of the frames from a group of frames) are passing the image quality assessment. In another example, a green dot or other indication, such as text, can appear on a display of the imaging device. The indication can be provided in real-time. An indication can be provided to the user when one or more features, diseases, or conditions are present or the probability for the presence of the features, diseases, or conditions. The indication can be provided in real-time.

Hierarchical Deep Learning Architecture with Feature Disentanglement

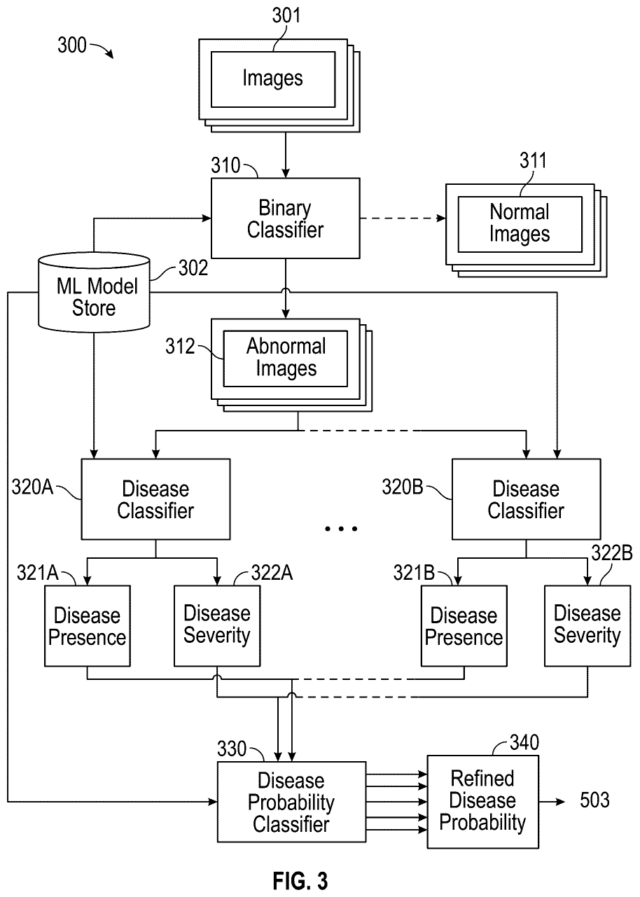

FIG. 3 is a block diagram of an example hierarchical deep learning architecture 300. The architecture can be implemented by one or more processors and one or more memories. In some cases, one or more memories 302 can store parameters of the classifiers illustrated in FIG. 3. In a first stage of the model, a binary classifier 310, such as a convolutional neural network (CNN-1), can classify the retinal image 301 as a normal image 311 or an abnormal image 312. If the retinal image is classified as a normal image 311, the processing can be completed. At the second stage, disease classifiers 320, such as through multiple CNN branches (CNN-2a, CNN-2b, . . . , CNN-2n) trained for specific diseases, can process the abnormal image 312. Separating the tasks of normal/abnormal classification and disease-specific classification into two stages can allow for each stage to be optimized for its specific task and thus better learn the task-specific features. The advantages of such approach can include the unique combination of the hierarchical architecture, feature disentanglement, exploitation of disease co-occurrence patterns, along with the end-to-end learning and optimization of the system.

In the initial filtering stage, the architecture of the binary classifier 310, such as via CNN-1, can be designed to differentiate between normal and abnormal retinal images. It can employ a combination of convolutional branches or layers, pooling branches or layers, activation functions, batch normalization, and fully connected branches or layers to learn hierarchical representations of the input abnormal image 312. The specific architecture of the neural network can vary, but the network can produce an “abnormal” or “normal” output, and if the output is “abnormal,” then the abnormal image 312 can be input in the second stage. If the output is “normal,” the system can stop.

In the second disease-specific stage, each branch of the second stage disease classifiers 320 can employ a dual-head architecture that allows for feature disentanglement, providing a CNN-2a as a disease classifier 320A for a particular disease, a CNN-2b as a disease classifier 320B for another particular disease, and so on. In some implementations, the number of branches in the disease classifiers 320 can be determined or predetermined based on user selection or determined by the system, such as being dependent on the picture taken or the determination of the first filter. In some implementations, the dual-head architecture can be a disease presence head, which can detect for disease presences 321 (shown as 321A and 321B), and a severity estimation head, which can detect for disease severities 322 (shown as 322A and 322B). The network can split into two fully connected layers after the last convolutional layer. One layer can be used for disease presences 321 and the other layer can be used for disease severities 322. This dual-task learning process can encourage the disease classifiers 320 to learn separate features for disease presence and disease severity, which can address both inter-disease and intra-disease variations. Each branch can then output a confidence score for the presence of its respective disease in the image.

The architecture 300 can provide one or more of the following advantages.

Exploiting Disease Co-Occurrence Patterns: The output of each disease classifiers 320 from stage two can then feed into a disease probability classifier 330. The disease probability classifier 330 can be a graphical model, such as a Conditional Random Field (CRF) or a Gaussian Conditional Random Field (G-CRF), to provide a correlation between different diseases and increase the accuracy of the predictions inputted into the graphical model to determine refined disease probabilities 340. The classifier 320 can determine the refined disease probabilities 340 by considering the non-linear co-occurrence patterns of different diseases. Compared to existing approaches, the hierarchical deep learning architecture 300 can provide significant benefits, including high detection accuracy, robustness to inter-disease and intra-disease variations, and the ability to exploit disease co-occurrence patterns effectively. These advantages can make it an effective tool for early and accurate detection of multiple diseases from retinal images. The disclosed system and methods can thus present a significant advancement in the field of retinal disease detection.

End-to-End Learning: An Expectation-Maximization (EM) algorithm, such as a Monte Carlo variant, can allow the hierarchical deep learning architecture 300 to optimize each and all its components simultaneously, which can enable efficient and robust training of the system despite the complexities introduced by the disease probability classifier 330. In some implementations, the backpropagation provided to update the hierarchical deep learning architecture 300 can occur after the refined disease probabilities 340 have been determined to analyze new retinal images 301 obtained by the imaging devices 240. This approach can also lead to more effective feature representations and more accurate predictions, providing significant clinical value.

### Multi-Branch Disease-Specific Disease Classifiers

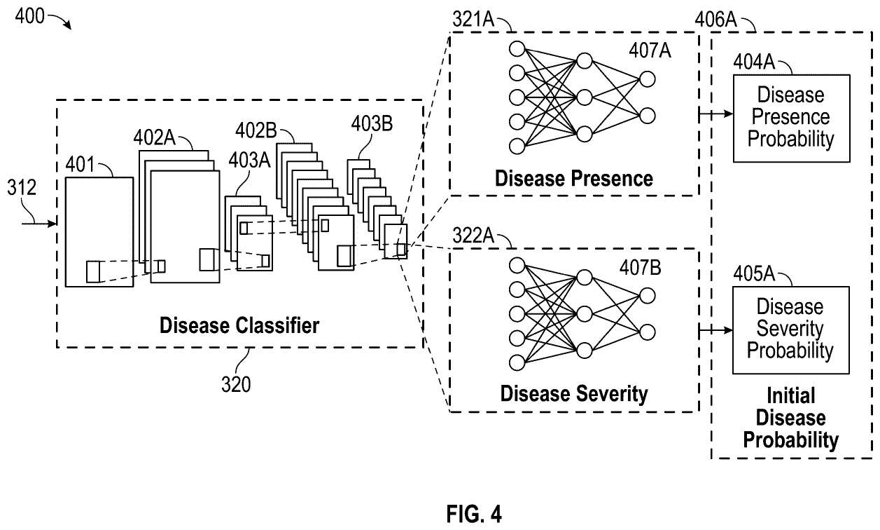

FIG. 4 is a block diagram 400 of a single, example disease classifier (such, as the disease classifier 320A or 320B) within the multi-branch stage two. In the second stage, the hierarchical approach can be split into multiple branches, and each branch as disease classifiers 320 can be designed and optimized to detect a specific retinal disease. Each branch can receive the same abnormal image 312 as a preprocessed input 401 but can have its unique set of parameters. The architecture of each disease-specific disease classifiers 320, each potentially as a CNN-2 model for the disease classifier 320A or 320B, can vary, but each CNN-2, by virtue of having its unique parameters, can learn to develop filters that are sensitive to the features of its specific disease, which can aid in the extraction of disease-specific features from the input image. Each CNN-2 can include convolution layers 402A, which generates a feature map by sliding a filter over the preprocessed input 401 to recognize patterns in the image based on the unique parameters to the particular disease, and pooling layers 403A, which down samples the feature map of convolution layers 402A, 402B to reduce overfitting. In some implementations, the disease classifiers 320 can include more than one convolution layers 402A, 402B and more than one pooling layers 403A, 403B. Each CNN-2 can employ a dual-head output architecture, where the final layer of each CNN-2 branch can feed into two separate fully connected layers 407: a fully connected layer 407A can be for disease presence 321A or 321A (a Detection Head), and a fully connected layer 407B can be for disease severity 322A or 322B (a Severity Head). A fully connected layer 407 connects the information extracted from the convolution layers 402A, 402B and pooling layers 403A, 403B and interconnects each input into the fully connected layer 407 to classify the input into a desired label. Each fully connected layer can serve a different purpose and can have its own output. In some instances, the output of detecting the disease presence 321A or 321B can be a disease presence probability 404A, and the output of estimating the disease severity 322A or 322B can be a disease severity probability 405A. The disease presence probability 404A and disease severity probability 405A can jointly be defined as the initial disease probability 406A for the particular disease that the disease classifier 320A or 320B is configured for.

Furthermore, the model can capture intra-disease variations effectively by passing the Detection Head's disease presence 321A or 321B output through a Sigmoid activation function for binary classification (presence or absence of a specific disease) while passing the Severity Head's disease severity 322A or 322B output through a softmax activation function for multi-class classification (severity levels). The feature disentanglement design can implement a multi-task learning regime where the disease classifier 320A or 320B model can be trained to simultaneously minimize the different loss functions of the Detection and Severity Heads. The total loss for each branch can be the sum of the two losses, and the network can be trained to minimize the total loss to encourage the disease classifier 320A or 320B model to learn feature representations that can be useful for both tasks. To prevent the model from ignoring one task in favor of the other, a regularization term could be introduced to balance the two loss terms and promote the model to learn a feature representation that can be useful for both tasks, as described further below in connection with end-to-end training.

In some implementations, this hierarchical learning approach can also be implemented using deep learning frameworks, such as TensorFlow or PyTorch. These frameworks can provide the building blocks (convolutional layers, activation functions, etc.) to construct the proposed CNN architectures and can support the end-to-end training of the model.

The hierarchical structure of the hierarchical deep learning architecture 300 focused on feature disentanglement can yield significant benefits to the multi-disease screening problem in one or more of the following ways.

The choice of a hierarchical deep learning architecture can delineate an explicit partitioning of the feature space. During training for the Al models, each stage of the model can be tuned to learn a different portion of the feature space that can correspond to a distinct task. The first CNN (CNN-1) for the binary classifier 310 can focus on learning features that can differentiate between a normal image 311 and abnormal image 312 of the retina. The second stage can consist of multiple CNN branches for the disease classifiers 320, and each can specialize in detecting a specific disease. The branches can target a more refined portion of the feature space that can relate to the unique characteristics of their corresponding diseases. Separating the first and second stages can impose an order in the feature space exploration, starting from the generic abnormalities down to disease-specific features. This ordered exploration can lead to a more organized and potentially less entangled feature space, which could boost the model's performance. Convolutional layers 402A, 402B in CNNs can operate by applying a set of learned filters over their preprocessed input 401 of the abnormal image 312 that can serve as feature detectors, where each filter can respond maximally to a specific type of feature in the input. By separating the classification tasks into two stages, the model can be encouraged to develop more selective filters at each stage. The filters in the first-stage CNN (CNN-1) can specialize in detecting generic abnormalities, while the filters in the disease-specific branches of the second stage can specialize in detecting features that can be characteristic of their respective diseases. This improved feature selectivity can lead to better performance in both detection of disease presences 321 and estimation of disease severities 322.

The dual-head architecture in each disease-specific branch of the disease classifiers 320 can enable the model to learn separate feature representations for disease presences 321 and disease severities 322 at an even finer level. The preceding convolutional layers 402A, 402B and pooling layers 403A, 403B shared by the detection head and severity head can extract common features. However, as the Detection Head and Severity Head can have separate sets of weights and can have their own loss function, they can learn to pay attention to different aspects of these shared features, potentially resulting in unique transformations through their respective fully connected layer 407. The implicit feature disentanglement can help the model to be more effective in both detection of disease presences 321 and estimation of disease severities 322, thus effectively capturing intra-disease variations.

The hierarchical structure focused on feature disentanglement can provide more stable training of the hierarchical deep learning architecture 300 because the weights determined by the dual-head architecture can be updated based on the gradients calculated through backpropagation in a deep learning model. The weights can be updated by propagating the error signal, such as the difference between the model's predictions and the actual labels, back through the network and incrementally adjusting the weights in a direction that can minimally reduce the error. In the proposed hierarchical architecture, the separation of normal/abnormal classification in stage one from disease-specific classification in stage two could potentially lead to more stable gradient propagation. In the first stage, the gradients could primarily reflect the generic characteristics of a normal image 311 vs. an abnormal image 312, potentially leading to a more stable training process as the model may not be influenced by the more nuanced variations within the abnormal category. In the second stage, the gradients could reflect disease-specific characteristics. The two-level gradient propagation could contribute to more stable network training, which in turn could lead to better generalization performance. This can be helpful for later analysis of a different abnormal image 312 analyzed by the hierarchical deep learning architecture 300, by potentially providing better trained disease classifiers 320 for more accurate initial disease probabilities 406A. This higher accuracy can be a result of less potential error for the detection of disease presences 321 to determine the disease presence probabilities 404A and for the estimation of disease severities 322 to determine the disease severity probabilities 405A.

Overall, the benefits of separating the two stages can include promoting more organized feature space exploration, improved feature selectivity, and stable gradient propagation, all of which can contribute to the superior performance of the model.

### Exploiting Disease Co-Occurrence Patterns

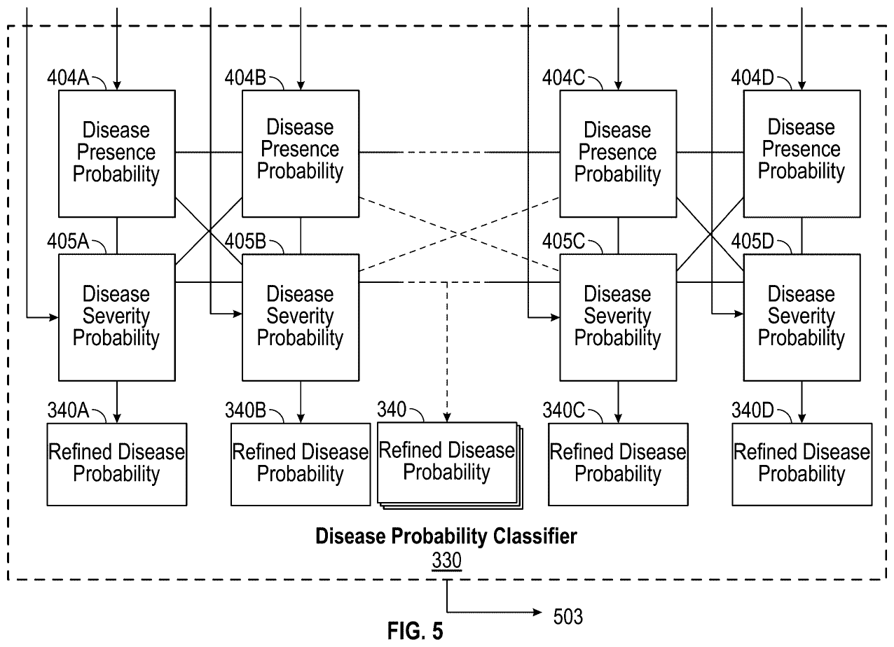

FIG. 5 is an example block diagram of the disease classifier 330 processing the initial disease probabilities 406A to determine refined diseases probabilities 340. The disease classifier 330, in some instances the third stage, can be a graphical model, such as a gaussian conditional random field (G-CRFs). Although the disclosure refers to G-CRF, similar to CNNs, it is not intended to be limiting and should be appreciated that the disclosed system and models can include others models in conjunction with or in lieu of. This third stage in the system can refine the initial disease probabilities 406A that were produced by the second-stage CNN-2 branches. The initial disease probabilities 406A can be the disease presence probabilities 404A and disease severity probabilities 405A of a particular disease that the corresponding disease classifiers 320 were designed for.

The relationships between different diseases, which can be disease co-occurrence patterns that are either predetermined or learned by the system, can be efficiently exploited using G-CRFs to connect each of the disease presence probabilities 404A and disease severity probabilities 405A. This innovative step that can allow the model to consider the complex and non-linear relationships between different diseases when generating its predictions. The G-CRF can be a structured probabilistic graphical model, which can model a joint probability distribution over retinal diseases as a Gaussian Markov random field.

In some instances, each node in the graph can represent the disease presence probability 404A, 404B, 404C, 404D and the disease severity probability 405A, 405B, 405C, 405D of a retinal disease, and each node can be represented as variables y=(y1, y2, . . . , yN), which can be predicted given a set of features x. These features x can represent the output feature vector of the represent the disease presence probability 404A, 404B, 404C, 404D and the disease severity probability 405A, 405B, 405C, 405D of the second-stage CNN branches. The attributes x=(x1, x2, . . . , xN) can interact with each node “yi” independently of one another. The edges can signify the conditional dependencies among the diseases. The generalized form of the conditional probability distribution of each node can be mathematically represented as:

$$P ( y \vert x , α , β ) = \frac{1}{Z ( x , α , β )} exp ( {\sum }_{i = 1}^{N} A ( α , {y}_{i} , {x}_{i} ) + I ( β , {y}_{i} , {y}_{j} ) )$$

Where A(α, yᵢ, xᵢ) can represent the association potential of the diseases selected per disease classifiers 320, which can model the relations between the predicted variables (yi), and the corresponding input vectors (xi), and I(β, yᵢ, yⱼ) can represent the interaction potential, which can model the relationship between nodes representing the initial disease probabilities 406A. Parameter Z(x, α, β) can represent a partition function. The association potential can be represented as:

$$A ( α , {y}_{i} , {x}_{i} ) = {\sum }_{k = 1}^{K} {{α}_{k} ( {y}_{i} - {R}_{k} ( {x}_{i} ) )}^{2}$$

Where Rₖ(xᵢ) can represent a predictor function returning yi given xi, for each node in the graph. This can be any predictor function, such as a regression function that outputs a prediction “yi” given a vector “xi.” K can represent the total number of predictor functions. The interaction potential can be defined as the following:

$$I ( β , {y}_{i} , {y}_{j} ) = - {\sum }_{i \neq j} {\sum }_{l = 1}^{L} {β}_{l} {{S}_{ij}^{l} ( {y}_{i} - {y}_{j} )}^{2}$$

Where Sᵢⱼ^{l} can be a similarity value between nodes i and j in the graph 1. L can be the total number of graphs, such as similarity functions. In some instances, we can have two graphs, with one for the disease severity 322A or 322B and one for the disease presence 321A or 321B.

The training of the graph can be defined as

$${arg max}_{y} P ( y \vert x , α , β ) ,$$

which can be achieved by optimizing over parameters alpha and beta by maximizing conditional log-likelihood:

$${arg max}_{α , β} {\sum }_{y} log P ( y \vert x , α , β )$$

For example, a node for disease severity probability 405A in the G-CRF for the stage three disease classifier 330 can accept the corresponding disease presence probability 404A from its corresponding CNN-2 disease classifiers 320A branch, along with the probabilities from neighboring nodes, such as the disease presence probability 404B and disease severity probability 405B from another disease classifier 320B for a different disease. Whether nodes are neighboring can be based on diseases that can co-occur. Each node can output a new, refined disease probability 340A with a higher accuracy than the original initial disease probability 406A, which can be because it better accounts for the non-linear interdependencies between diseases. Similarly, the refined disease probability 340A can include a new probability for the disease presence probability 404A by using the corresponding disease severity probability 405A and neighboring nodes. This process can produce refined diseases probabilities 340 for each disease analyzed by the second stage. For example, a refined disease probability 340A, 340B, 340C, 340D can be determined by running each of the disease presence probability 404A, 404B, 404C, 404D and disease severity probability 405A, 405B, 405C, 405D through the third stage.

The use of G-CRFs to model and exploit disease co-occurrence patterns in retinal images can contribute to the enhanced performance of the hierarchical deep learning architecture 300 and can allow the system to account for complex disease interdependencies in the final predictions, which can improve the overall accuracy and reliability of the disease detection process by exploiting the co-occurrence patterns of diseases in retinal images. A graphical model such as the G-CRF can effectively represent complex relationships between different diseases. In some instances, each node in the G-CRF can represent a different retinal disease, and the edges between nodes can represent the conditional dependencies or co-occurrence relationships between these diseases. The disease presence probabilities 404A of each disease and corresponding disease severity probabilities 405A can be adjusted based on these relationships, providing a nuanced and interconnected view of disease occurrence that individual CNN-2 branches cannot achieve in isolation.

The advantages of the third stage can be illustrated with an example scenario, using the case of diabetic retinopathy (DR) and glaucoma. Given that the underlying process causing DR, namely diabetes, can also increase the likelihood of glaucoma, the two diseases can often co-occur in patients. However, the presence of glaucoma in a retinal image may not necessarily mean an increased likelihood of DR. Suppose that the CNN-2 branch corresponding to glaucoma initially gives a disease presence probability 404A of 0.2, while the DR CNN-2 branch outputs a disease presence probability 404B of 0.9. In a system without the third stage, which may be a G-CRF, these probabilities may be considered independently, which can lead to a low likelihood prediction of glaucoma. However, in a system with G-CRF, these initial probabilities may not be the final word. The G-CRF, which models the co-occurrence patterns, can adjust the initial disease probabilities 406A.

Suppose the similarity value between DR and Glaucoma nodes is 0.7, reflecting a significant positive correlation. The high DR disease presence probability 404B can influence the Glaucoma disease presence probability 404A, and the Glaucoma presence probability of the refined disease probability 340A can be adjusted upwards. Assuming the final Glaucoma presence probability of the refined disease probability 340A is a weighted average of the initial disease probability 406A and the DR disease presence probability 404B and DR disease severity probability 405B, with weights determined by the similarity value, the final Glaucoma presence probability of the refined disease probability 340A could be: 0.7*0.9 (DR disease presence probability 404B)+0.3*0.2 (initial Glaucoma disease presence probability 404A)=0.69. This adjusted presence probability of the refined disease probability 340A is substantially higher than the initial probability of 0.2, demonstrating how G-CRF's exploitation of real-world disease co-occurrence patterns can enhance the accuracy of disease predictions. In this way, the system can accurately predict a high likelihood of both DR and Glaucoma, thereby reflecting the previously determined disease co-occurrence pattern.

This difference between a glaucoma presence probability of 0.2 and 0.69 could potentially alter the clinical decision-making process in a significant way. The presence probability can represent the model's confidence in its prediction, and a higher probability could prompt more decisive and immediate actions. A clinician might consider a Glaucoma disease presence probability 404A of 0.2 relatively low risk. Depending on the clinician's judgment and the patient's symptoms, they might adopt a more conservative approach, perhaps recommending closer monitoring and regular check-ups to track the development of the condition, but not necessarily initiating immediate treatment. However, a clinician could perceive a Glaucoma presence probability of 0.69 as a substantial risk. Given glaucoma's potential to cause irreversible vision loss, the clinician might choose to take immediate action. The clinician can order a more in-depth evaluation of the patient and possibly prescribe medication to decrease intraocular pressure, or the clinician can even recommend laser treatment or surgery, depending on the specific patient's circumstances and the severity of glaucoma. Additionally, given the co-occurrence of glaucoma with diabetic retinopathy, the clinician might order additional tests to assess the patient's blood sugar levels, vascular health, and other related parameters. Thus, a higher disease probability can indicate more urgency for medical management and can lead a clinician to take comprehensive preventative and therapeutic actions.

In sum, the innovative application of the third stage (such as, G-CRF) can allow the system to consider complex and non-linear relationships between different diseases when generating predictions, increasing the ability of the model to accurately predict disease probability, which can directly impact clinical decision-making and patient outcomes.

### End-to-End Learning Approach

The system can use a full end-to-end learning approach to allow components, from the hierarchical deep learning architecture to the G-CRF exploitation of disease co-occurrence patterns, to work together to improve the final output. The model can learn to optimize each and all parameters across all layers simultaneously using backpropagation for the CNNs and Expectation maximization for the G-CRF model, which can ensure that the model learns the most effective feature representations at all layers for the given tasks and can ensure a system-wide optimization towards the best possible disease prediction outcomes.

In order to optimize the graph-based structure of the G-CRF model, the system can employ a variant of the Expectation-Maximization (EM) algorithm, such as the Monte Carlo EM algorithm. The EM algorithm can be a two-step process that can be performed iteratively. The EM algorithm can first compute the expected log-likelihood under the current parameter estimates and can second maximize this expected log-likelihood to update the parameters. The E-step can involve sampling from the current estimate of the posterior distribution P(Y|X).

The Monte Carlo EM algorithm can address the challenge of intractable integrals in the E-step by approximating the expected log-likelihood through sampling. Multiple loss functions can be optimized system-wide in the end-to-end learning method that can be used to train this model. For the multi-task learning scenario, each head of the second-stage CNN (CNN-2) can be equipped with its own loss function: binary cross-entropy for disease detection and categorical cross-entropy for severity estimation. In some instances, the loss function for disease detection can be referred to as binary cross-entropy loss function and the loss function for severity estimation can be referred to as a categorical cross-entropy loss function.

Binary cross-entropy can be given by the following equation:

$$BCE = \frac{1}{N} {\sum }_{i = 1}^{N} {y}_{i} * log ( p ( {y}_{i} ) ) + ( 1 - {y}_{i} ) * log ( 1 - p ( {y}_{ii} ) ) ,$$

where yi can be the true label and p(yi) can be the predicted probability.

Categorical cross-entropy can be defined as the following equation:

$$CCE = - {\sum }_{i = 1}^{C} {y}_{{i}_{c}} log ( {P}_{{i}_{c}} ) ) ,$$

where the sum can be over all classes “C,” y_i can be the true label for class i, and p_i can be the predicted probability for class i.

The G-CRF can incorporate an additional loss function that can measure the discrepancy between the model's predictions of disease co-occurrence patterns and the actual patterns in the data. In some instances, this can be referred to as a co-occurrence loss function. The total loss of the system, which it can seek to minimize during training, can be a weighted sum of these individual losses, where weights a, 3, and y can be determined based on validation performance:

$$L\_total = αL\_detection + βL\_severity + γL\_GCRF$$

This end-to-end learning approach can allow the model to optimize all its components simultaneously, taking into account the complex interactions between these components. This approach can lead to more effective feature representations and more accurate predictions, providing significant clinical value.

### Results Display

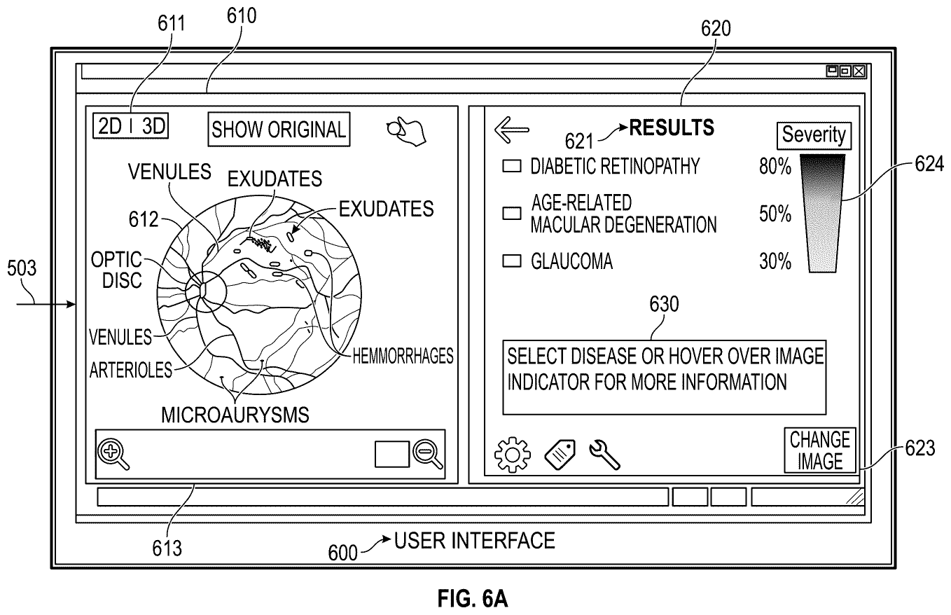

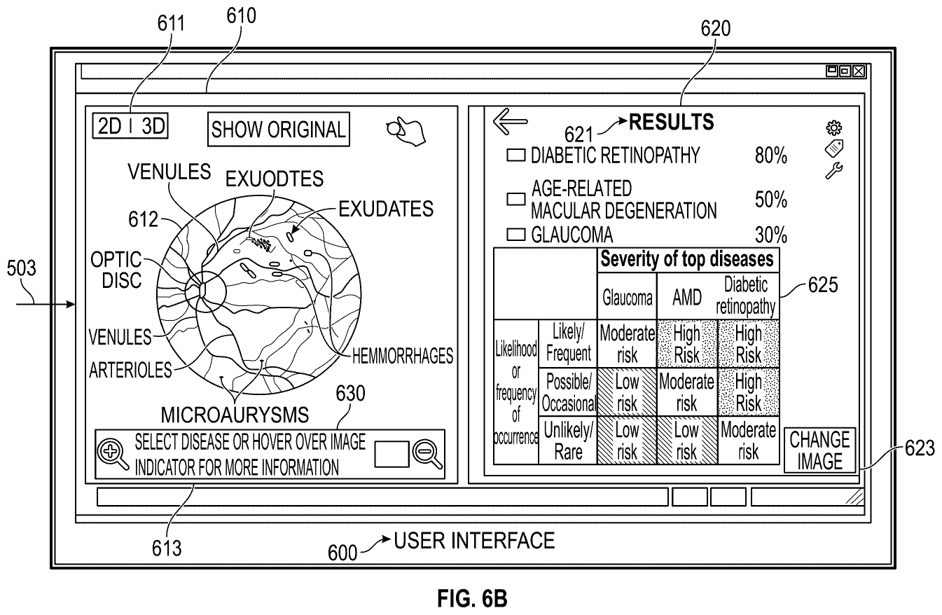

FIG. 6A and FIG. 6B are example user interfaces of a results display 600 within the hierarchical deep learning architecture 300, which illustrate an indication of the presence and severity of diseases from an abnormal image 312, where the severity is displayed in a different manner. The results display 600, outputted at 503 from the classifier 330, can illustrate a comprehensive report of potential disease indicators, each linked with the corresponding abnormal image 312 captured by the imaging devices 240. After the hierarchical deep learning architecture 300 has processed the images and produced either results for initial disease probabilities 406A or refined disease probabilities 340, these results can be presented to the clinician on the results display 600. The results display 600 can be implemented on a user device, such as the imaging devices 240 or retina camera 100, or on a separate computing device. The results display 600 can provide users or clinicians with an easy-to-understand representation of the patient's condition.

The results display 600 can be a graphical user interface (GUI) that can be implemented on a standalone computer monitor, a portable device like a tablet, or even a web-based interface that can be accessed from any internet-connected device. The primary goal can be to present the results in a clear, intuitive, and actionable format for the clinician. The GUI can be designed with user experience (UX) principles in mind, ensuring that clinicians can easily understand and act upon the results. For example, positive detections of the selected diseases might be highlighted in a distinct color or accompanied by an alert symbol.

The results display 600 can also allow a user (such as, a clinician) to interact with the analyzed images 610 (for instance, analyzed retinal images) and the results 620. For instance, the user may be able to zoom in or out of images 613, rotate 3D images 611, adjust image brightness or contrast, or click on areas or regions of interest 612 for more information. The display can show the original images with relevant areas highlighted for the regions of interest 612, probability indicators 621, and an informational box for diseases 622. Furthermore, the user can shuffle between different images 623. This information aids the user in confirming the diagnosis and planning the patient's subsequent treatment steps.

Within the results 620, the probability indicators 621 can illustrate either the disease presence probabilities 321 or the presence probabilities of the refined disease probabilities 340 of the diseases analyzed by the disease classifiers 320. In some instances, the ordering of the disease can be based on the highest likelihood of disease presence. In some instances, an information box for diseases 630 can either be displayed in the results 620, as shown in FIG. 6A, or in the analyzed images 610 section, as shown in FIG. 6B. As shown in FIG. 6A, the disease severity probabilities 405A or the severity probabilities of the refined disease probabilities 340 can be illustrated as a gradient scale 624 corresponding to the probability indicators 621. In some instances, the ordering of the diseases can correspond to the highest to least severity probability and the gradient of the gradient scale 624. The gradient scale 624 can be colored. For example, higher severity can be shown in red, medium severity can be shown in yellow, and lower severity can be shown in green or blue colors.

As shown in FIG. 6B, the disease severity probabilities 405A or the severity probabilities of the refined disease probabilities 340 can be illustrated as a risk analysis box or table 625 that depicts the level of severity for selected diseases based on the likelihood or frequency of occurrence. In some instances, the selected diseases can be the diseases with the highest presence probability. The table 625 can be colored. For example, higher severity (such as “High risk”) can be shown in red, medium severity (such as, “Moderate risk”) can be shown in yellow, and lower severity (such as, “Low risk”) can be shown in green or blue colors.

When a retinal image has been classified as normal, the results display 600 can include a label, such as “Normal.” Such label can be included in the results 620 section.

This comprehensive diagnostic imaging system can either be a standalone solution or integrated within a broader health information system. Ultimately, this system can represent a significant advancement in the field of medical imaging and diagnostics.

### Terminology

All of the methods and tasks described herein can be performed and fully automated by a computer-implemented system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid-state storage devices, disk drives, etc.). The various functions disclosed herein can be embodied in such program instructions or can be implemented in application-specific circuitry (e.g., ASICs or FPGAs) of the computer system. Where the computer system includes multiple computing devices, these devices may, but need not, be co-located. The results of the disclosed methods and tasks can be persistently stored by transforming physical storage devices, such as solid-state memory chips or magnetic disks, into a different state. In some embodiments, the computer system can be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, multiple processors or processor cores, or on other parallel architectures rather than sequentially

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application-specific integrated circuit (ASIC), a field programmable gate array (FPGA) or other programmable logic devices, discrete gate or transistor logic, discrete hardware components, or any combination thereof designed to perform the functions described herein. For example, a processor device can be a microprocessor, but in the alternative, the processor device can be a controller, microcontroller, or logic circuitry that implements a state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device can also include primarily analog components. For example, some or all of the rendering techniques described herein can be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, a software module executed by a processor device, or a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. Alternatively, the storage medium can be integral to the processor device. For example, the processor device and the storage medium can reside in an ASIC. The ASIC can reside in a user terminal. Alternatively, the processor device and the storage medium can reside as discrete components in a user terminal.

The conditional language used herein, such as, among others, “can,” “can,” “might,” “may,” “e.g.,” and the like, unless specifically stated otherwise or otherwise understood within the context as used, is generally intended to convey that certain embodiments include, while other embodiments do not include certain features, elements or steps. Thus, such conditional language is not generally intended to imply that features, elements, or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive languages such as the phrase “at least one of X, Y, or Z,” unless specifically stated otherwise, is otherwise understood with the context as used in general to present that an item, term, etc., can be either X, Y, or Z, or any combination thereof (e.g., X, Y, or Z). Thus, such disjunctive language is not generally intended to, and should not, imply that certain embodiments require at least one of X, at least one of Y, and at least one of Z to each be present.

While the above-detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. Accordingly, the scope of certain embodiments disclosed herein is indicated by the appended claims rather than by the foregoing description. All changes which come within the meaning and range of equivalency of the claims are to be embraced within their scope.

What is claimed is:

1. A non-transitory computer readable storage medium storing instructions that, when executed by one or more processors, cause the one or more processors to perform a method for disease detection using retinal images, the method comprising:
processing a retinal image of a retina with a first machine learning model to determine whether the retinal image reflects an abnormal condition or a normal condition of the retina;
at a first time, determining that the retinal image reflects the abnormal condition of the retina and in response to determining that the retinal image reflects the abnormal condition of the retina:
processing the retinal image by a plurality of second machine learning models, wherein each machine learning model of the plurality of second machine learning models is configured to identify 1) a presence of a disease different from any other machine learning model of the plurality of second machine learning models and 2) a severity of the disease;
processing a plurality of disease presences and a plurality of disease severities determined by the plurality of second machine learning models with a third machine learning model to determine a plurality of disease indicators based on relationships between a plurality of diseases and their respective severities identified by the plurality of second machine learning models;
outputting the plurality of disease indicators;
outputting the retinal image; and
at a second time, determining that the retinal image reflects the normal condition of the retina and in response to determining that the retinal image reflects the normal condition of the retina, outputting the retinal image.

2. The non-transitory computer readable storage medium of claim 1, wherein the relationships between the plurality of diseases comprises a disease co-occurrence pattern, and wherein the third machine learning model comprises a graph-based machine learning model configured to determine the plurality of disease indicators using the disease co-occurrence pattern.

3. The non-transitory computer readable storage medium of claim 2, wherein the graph-based machine learning model comprises conditional random fields.

4. The non-transitory computer readable storage medium of claim 2, wherein the plurality of disease indicators is determined by:
generating a plurality of nodes, wherein each node of the plurality of nodes is based on a plurality of detection probabilities for the plurality of disease presences and a plurality of severity estimation probabilities for the plurality of disease severities;
determining one or more neighboring nodes for each node of the plurality of nodes based on the disease co-occurrence pattern; and
updating a detection probability and a severity estimation probability for each disease of the plurality of diseases based on the plurality of detection probabilities and plurality of severity estimation probabilities for neighboring nodes, the plurality of disease indicators comprising the updated detection probabilities and the updated severity estimation probabilities.

5. The non-transitory computer readable storage medium of claim 1, wherein the first machine learning model comprises a binary classification convolutional neural network configured to differentiate between abnormal and normal conditions of the retina.

6. The non-transitory computer readable storage medium of claim 1, wherein the plurality of second machine learning models comprises a plurality of multi-branch disease-specific convolutional neural networks.

7. The non-transitory computer readable storage medium of claim 1, wherein each machine learning model of the plurality of second machine learning models includes a disease detection branch and a disease severity branch that is separate from the disease detection branch.

8. The non-transitory computer readable storage medium of claim 7, wherein the disease detection branch is configured to determine a detection probability for the presence of the disease, and wherein the disease severity branch is configured to determine a severity estimation probability for the severity of the disease.

9. The non-transitory computer readable storage medium of claim 7, wherein the disease detection branch and the disease severity branch are configured to minimize a loss function using backpropagation to reduce a combination of the loss function for the disease detection branch and the loss function of the disease severity branch.

10. The non-transitory computer readable storage medium of claim 9, wherein:
the third machine learning model is configured to minimize a loss function that measures a discrepancy between a predicted co-occurrence pattern and an actual co-occurrence pattern, the loss function for the third machine learning model comprising a co-occurrence loss function.

11. The non-transitory computer readable storage medium of claim 10, wherein:
the loss function of the disease detection branch comprises a binary cross-entropy loss function and the loss function of the disease severity branch comprises a categorical cross-entropy loss function.

12. The non-transitory computer readable storage medium of claim 10, wherein the method further comprises minimizing a total loss determined based on a weighted sum of weights for each of the loss functions and updated weights determined based on a validation performance for each of the loss functions.

13. The non-transitory computer readable storage medium of claim 12, wherein the updated weights are determined based on backpropagating an error signal corresponding to each of the loss functions to minimize the error signal.

14. The non-transitory computer readable storage medium of claim 1, wherein the method further comprises:
optimizing the plurality of second machine learning models using backpropagation; and
optimizing the third machine learning model using an expectation maximization.

15. The non-transitory computer readable storage medium of claim 1, wherein outputting the plurality of disease indicators comprises outputting probabilities of the plurality of disease severities.

16. The non-transitory computer readable storage medium of claim 15, wherein outputting probabilities of the plurality of disease severities comprises outputting an ordered representation of probabilities of the plurality of disease severities.

17. The non-transitory computer readable storage medium of claim 15, wherein outputting probabilities of the plurality of disease severities comprises outputting a level of severity for a plurality of diseases based on a likelihood or frequency of disease occurrence.

18. A method for disease detection using retinal images, the method comprising:
processing a retinal image of a retina with a first machine learning model to determine whether the retinal image reflects an abnormal condition or a normal condition of the retina;
at a first time, determining that the retinal image reflects the abnormal condition of the retina and in response to determining that the retinal image reflects the abnormal condition of the retina:
processing the retinal image by a plurality of second machine learning models, wherein each machine learning model of the plurality of second machine learning models is configured to identify 1) a presence of a disease different from any other machine learning model of the plurality of second machine learning models and 2) a severity of the disease;
processing a plurality of disease presences and a plurality of disease severities determined by the plurality of second machine learning models with a third machine learning model to determine a plurality of disease indicators based on relationships between a plurality of diseases and their respective severities identified by the plurality of second machine learning models;
outputting the plurality of disease indicators;
outputting the retinal image; and
at a second time, determining that the retinal image reflects the normal condition of the retina and in response to determining that the retinal image reflects the normal condition of the retina, outputting the retinal image,
wherein the method is performed by one or more processors.

19. The method of claim 18, wherein the relationships between the plurality of diseases comprises a disease co-occurrence pattern, wherein the third machine learning model comprises a graph-based machine learning model configured to determine the plurality of disease indicators using the disease co-occurrence pattern, and wherein the plurality of disease indicators is determined by:
generating a plurality of nodes, wherein each node of the plurality of nodes is based on a plurality of detection probabilities for the plurality of disease presences and a plurality of severity estimation probabilities for the plurality of disease severities;
determining one or more neighboring nodes for each node of the plurality of nodes based on the disease co-occurrence pattern; and
updating a detection probability and a severity estimation probability for each disease of the plurality of diseases based on the plurality of detection probabilities and plurality of severity estimation probabilities for neighboring nodes, the plurality of disease indicators comprising the updated detection probabilities and the updated severity estimation probabilities.

20. The method of claim 18, wherein:
each machine learning model of the plurality of second machine learning models includes a disease detection branch and a disease severity branch that is separate from the disease detection branch;
the disease detection branch is configured to determine a detection probability for the presence of the disease;
the disease severity branch is configured to determine a severity estimation probability for the severity of the disease; and
the disease detection branch and the disease severity branch are configured to minimize a loss function using backpropagation to reduce a combination of the loss function for the disease detection branch and the loss function of the disease severity branch.
